(19) United States
(12) Patent Application Publication
(10) Pub. No.: US 2003/0060716 A1
(43) Pub. Date: Mar. 27, 2003

(54) Device for inserting the distal end of a hollow needle in a blood vessel

(76) Inventor: Jens Peter Heidrich, Lubeck (DE)

Correspondence Address: MATTHEW R. JENKINS, ESQ., 2310 FAR HILLS BUILDING, DAYTON, OH, 45419, US

(21) Appl. No.: 10/163,252

(22) Filed: Jun. 5, 2002

(30) Foreign Application Priority Data
Dec. 1, 2000 PCT/DE00/04310
Dec. 6, 1999 (DE) 199 58 688.8

(51) Int. Cl. A61M 5/20

(52) U.S. Cl. 600/464

(57) ABSTRACT

The device (1) is provided for introducing an elastic indwelling cannula (15) with a rigid puncture cannula located therein, into a blood vessel of a human or animal body. It comprises holders (8, 13) for the cannulas (9, 15) to be introduced as well as a locating means for locating the vessel and for aligning the cannulas (9, 15) to the vessel. The holders (8, 13) are in each case fastened on the device by way of a guide (10) and displaceably mounted in the introduction direction as well as in the counter direction. They may be traversed electromotorically independently of one another along the guides. The control of the electromotors is effected in dependence on the signals of the locating means so that the complete introduction procedure may be automatically controlled up to the retraction of the puncture cannula.

### BACKGROUND OF THE INVENTION

The invention relates to a device for introducing an elastically bendable indwelling cannula together with an essentially rigid puncture cannula located therein into a blood vessel of a human or animal body.

The introduction of cannulas into blood vessels is one of the most common actions of every physician. Although this is carried out daily many, many times, with this there often occur problems, and specifically on piercing in the cannulas which are to be considered as essentially rigid, as they are for example provided with syringes, as well as in particular with large-lumen indwelling cannulas which consist of a rigid part for puncturing and a bending-elastic part for the subsequent dwelling in the vessel. The manual methods common up to now are to a great extent dependent on the subjective assessment and the skill of the operating physician of locating and hitting the blood vessel. Often particular anatomical situations, adipositis or pathological conditions such as for example edemas create problems. On account of the prevailing size conditions and tissue characteristics, the introduction of indwelling cannulas is particularly difficult with babies and infants. One may only approximate the course of the veins. Although devices have been suggested which are to simplify the location and introduction of a syringe cannula, they have not succeeded in practice and are not so suitable for applying indwelling cannulas.

From U.S. Pat. No. 5,311,871 there is known a document with which an ultrasound producer is arranged in the plunger base of a syringe. The oscillations produced here are transmitted to the cannula via the fluid located in the syringe cylinder, wherein the emitter is simultaneously the receiver of the reflected sound waves and Doppler sonography is applied in order to locate a blood vessel. A similar arrangement is known from U.S. Pat. No. 5,080,103. These locating aids have however not succeeded in practice, since the locating principle only functions when the cannula has intimate contact with the body, i.e. has already penetrated through the surface of the body. They are accordingly unsuitable for vessels running close under the skin.

From U.S. Pat. No. 5,427,108 there is known a device with which a blood vessel may likewise be located using ultrasound Doppler sonography, wherein the device at the same time is aligned at a direction to the vessel such that the cannula may be manually introduced through a guide of the device into the vessel. A device known from U.S. Pat. No. 4,527,569 is even more mechanised and with this a holder for the cannula to be introduced is largely positioned automatically with regard to the located blood vessel and the introduction of the cannula into the vessel may then be effected in a manually controlled manner. The two last-mentioned devices are technically complicated and have likewise not been successful in practice. Although the location of vessels may be simplified with such devices, the introduction of the cannulas themselves is however hardly simplified at all since the physician must estimate the course of the vessel and therefore himself must estimate the penetration depth.

From DE 41 42 795 C1 there is known a device for injecting or puncturing joint cavities with which a puncture syringe is mounted in a syringe holder which is electromotorically traversable on a carrier. The motor control is effected dependent on the signal of a sonar measuring device in a manner such that the correct penetration depth of the cannula arranged on the syringe is always ensured. Notwithstanding the fact that this device is provided for puncturing joint cavities, it is not suitable for introducing cannulas and even less suitable for introducing indwelling cannulas into vessels, since they introduce the needle essentially perpendicular to the body surface, and only the penetration depth is determined. Furthermore the ultrasound measurement head surrounding the cannula is not so suitable for vessel puncture since it completely covers the penetration location on the skin and thus prevents a visible control which usefully should be carried out continuously.

From DE 42 06 065 C2 there is known an auxiliary device for introducing a canulla of a syringe with which there are provided two sound heads so that the position and course of the vessel to be punctured may be determined quite reliably. Here it is the case however of a pure location device, a mechanisation of the puncture procedure is not provided with this device so that the actual puncture procedure is not reproducible and as before is subject to the manual skill of the physician.

The device known from DE 94 14 727 U1 serves for biopsy, thus for removing tissue samples and is therefore not applicable to the field of application being discussed here.

Common to all previously mentioned devices is the fact that they merely represent auxiliary devices on introducing a cannula into a vessel and therefore do not yield any reproducible results or are only provided for the mechanised introduction of simple cannulas, thus are not suitable for introducing indwelling cannulas in which two cannulas are to be introduced and then are to be moved relative to one another in a designated manner.

### BRIEF SUMMARY OF THE INVENTION

Against this state of the art it is the object of the invention to provide a device for introducing an elastically bendable indwelling cannula with an essentially rigid puncture cannula, which largely mechanises and automises the laying of an indwelling cannula.

This object according to the invention is achieved by the features specified in claim 1. Advantageous embodiments of the invention are specified in the dependent claims, and explained in the subsequent description and drawings.

In order to make the reliability and reproducibility of the introduction procedure as independent as possible of the manual skill and the prevailing anatomical particularities, the invention not only envisages the provision of a location means as an aid for locating a vessel and a holder for the cannulas to be introduced, but also envisages mechanising the complete introduction procedure in that for the indwelling cannula and for the puncture cannula in each case there is provided a separate holder, which are traversable along a guide independently of one another in the direction of introduction as well as in the counter direction. The control is effected dependent on the exit signal of the location means which is designed for locating the vessel as well as for determining the course of the vessel, and specifically in front of and behind the puncture location or the expected puncture location of the puncture cannula. Since the holders are traversable independently of one another, not only may the indwelling cannulas with the puncture cannula located therein be introduced into the vessel, but furthermore the puncture cannula may be retracted and at the same time the indwelling cannula where appropriate may be advanced further, as is usual also with the manual laying of such indwelling cannula.

Basically with the device according to the invention simple cannulas may also be introduced into a vessel in a secure and reproducible manner, however the application for laying indwelling cannulas is particularly advantageous. Indwelling cannulas in comparison to syringe cannulas have a considerably larger lumen so that it is even more important for this cannula to be brought into the designated position precisely and quickly, since pain, dangers of injury and their consequences are significantly greater on introducing indwelling cannulas than with other cannulas. Apart from the complete mechanisation of the introduction procedure the device according to the invention further offers the advantage that on account of the technological control of the connection between the exit signal of the location means and the drive means for introducing the cannulas, one may securely reach blood vessels lying even deeper. At the same time by way of a suitable coordination of the introduction speed as well as by way of the fact that one effects the introduction always at a favorable angle, the patient does not feel so much pain. Furthermore by way of the secure and very exact introduction, the danger of injury to the wall of the vessel and the consequences of this such as bleeding, haematoma or inflammation of the vessel is considerably reduced. Finally by way of machanising the introduction procedure the risk of infection to the operating person is reduced since the manual introduction of the sharp cannulas during puncture is avoided and the distance to the patient is increased so that the operating person does get into contact with spurting blood so easily.

Preferably the device has two locating devices, which are designated such that the vessel to be punctured is located in a manner such that the puncture location and a region in the puncture direction behind the puncture location is detected so that the complete introduction procedure may be effected according to the course of the vessel with location control. The locating means however also determine the region in front of the puncture location so that the course of the vessel is detected in the direct vicinity of the puncture location or the puncture location to be expected is detected and also monitored during the introduction procedure.

As a location means there serves an ultrasound emitter and receiver which preferably functions Doppler-sonographically, since then on account of the flow within the vessels this may then be particularly differentiated from other organs and furthermore on account of the flow direction one may without further ado ascertain whether with the vessel which has just been located, it is the case of a vein or an artery. Such ultrasound locating systems have been known for a long time, and function either continuously (CW Doppler sonography) or also in a pulsed manner (PW Doppler sonography). They are available on the market and therefore are not described individually here. In this context one refers to Schaeberle, Wilhelm, Ultrasound in vessel diagnosis, Springer publishing house, Berlin, Heidelberg 1998 (ISBN 3-540-631148-8).

In order to ensure that the introduction of the cannula is only effected when the device is located in the designated position with respect to the vessel to be punctured, the control is designed such that the drive moving the cannulas in the introduction direction is only switched when the evaluation of the signals emitted by the locating means by way of the control results in the fact that the device is located in a position to the vessel to be punctured which is suitable for the puncture procedure. In order to provide the physician with an aiding position on aligning the device to the patient, there is provided an optical or acoustic display which emits a signal when the device is located in the designated position for puncture of the located vessel. Such a display may for example be formed by light diodes which by way of an optical signal in a program-controlled manner indicate when the optimal position of the device to the vessel is reached for introducing the cannula. For this the device is moved manually over the skin until the display lights up.

In order to simply a course orientation of the device on application, it is useful for the distal end of the device to be equipped with a contact surface which is set obliquely to the direction of the traversing direction of the guides, thus to the movement direction of the cannula holders. The angle of this oblique position is usefully between 30° and 45°, thus in the region in which an optimal alignment of the cannulas with regard to the vessel to be punctured is to be expected when this contact surface is brought to rest on the skin of a patient, for example in the region of the extremities. It is to be understood that this angle may be adapted to the shape of the distal end of the cannula. In order however to provide the user with the possibility of aligning the device with regard to the located vessel in spite of the two dimensional contact, it is useful not to form the contact surface completely flat, but to form it crowned so that at least in the borders to be expected the device may be pivoted forward, backwards or also to the one or other side. The crowned design also simplifies the sliding over the skin for locating the vessel. In particular with the application of ultrasound locating means it is necessary for there to exist a good coupling between the emitter and the object to be located or between the receiver and the object to be located, i.e., a connection conducting sound waves well. In order to achieve this it is useful to arrange the emitter-receiver arrangement on the rear side of the contact surface.

It is useful to provide two linear guides, specifically one for the puncture cannula and a further one arranged parallel thereto for the indwelling cannula. With this the drives may be activated separately in order also to be able to carry out the procedure of the retraction of the puncture cannula as well as the advancing of the indwelling cannula in a largely mechanised manner.

Commercially available indwelling cannulas for puncturing as a rule have a rigid, sharply ground puncture cannula which is surrounded by the actual blunt, elastically bendable indwelling cannula which together with the puncture cannula is introduced into the vessel. After puncture the indwelling cannula is advanced further in the introduction direction and the puncture cannula is retracted and finally is removed manually.

The device may have different shapes according to the application purpose. An approximately pistol-shaped design of the device has shown to be particularly advantageous, with which the release button is provided in the front part of the grip piece and the control and evaluation electronics are preferably likewise integrated in the grip piece. The electrical energy required for the operation of the device is preferably made available by a battery arranged in the grip piece or another suitable location, wherein as is usual with such apparatus one may provide a charging holder or also an external charging station for the battery. Such an arrangement is particularly useful when the device is to be held and guided with one hand. The pistol-like configuration is selected in order to be able to puncture as flexibly as possible every vessel reachable on the torso or extremities. The actuation with only one hand gives the user the possibility at any time of holding the extremity of the patient with the other hand and where appropriate of intervening to correct or support. After applying the indwelling cannula the free hand is required to release the holders from the indwelling cannula and to remove the puncture cannula and to set up suitable conduit connections for example for an infusion.

The holders for the puncture cannula, which are provided on the guides, and for the indwelling cannula must be provided with drive means. With this it is to be seen as particularly advantageous to arrange the guides to lie outside the housing and to set the actual drive inwards in the pistol grip and to merely let one lug via a longitudinal recess engage in the piston-like body for the purpose of the drive connection. This is useful already for reasons of hygiene since all components which come into contact with the cannulas are accessible and removable for the purpose of cleaning and disinfection. The drive at the same time is effected preferably by way of electric motors which for example drive a worm into which a lug connected to the holder engages, so that the holder is traversable in a defined manner along the guide by way of selecting the rotational direction and number of rotations of the motor. In place of a worm drive one may also employ a telescopic drive. Such drives are known for example for extending and retracting telescopic car antennas.

In order to ensure that the cannulas are introduced in a designated manner, according to their constructional type and size, it is useful to provide suitable sensorics on the device for detecting the cannula type and/or size. Since the cannulas usually with a lumen increasing in size may also increase in their constructional length, the cannula size used in each case may be determined by a row of photo-diodes which are attached on the upper side and which function in the manner of a reflective light barrier, and this transmitted to the control. Alternatively there may also be provided a scanner which detects a bar code attached on the side of the cannula, in which the cannula type and the cannula size are specified in a coded manner. A mechanical detection is also conceivable, either for the case that a device is designed only for the application of a certain cannula type or that the size differences are mechanically sensed.

### BRIEF DESCRIPTION OF THE DRAWINGS

The invention is hereinafter explained by way of an embodiment example shown in the drawings. There are shown in:

FIG. 1 in a schematic representation, a lateral view of a device according to the invention,

FIG. 2 a plan view of the device according to FIG. 1,

FIG. 3 a view of a common indwelling cannula with a puncture cannula,

FIG. 4 a view, displaced by 90° with respect to the cannula axis, of the puncture cannula according to FIG. 3,

FIG. 5 the indwelling cannula in a representation according to FIG. 4,

FIG. 6 the arrangement of the distal end of the device with the location means in relation to a vessel which is to be punctured,

FIG. 7 a schematic, perspective representation of the device on application on an arm,

FIG. 8 the arrangement of the distal end of the device at the beginning of the introduction procedure and

FIG. 9 the arrangement according to FIG. 8 during the advance of the indwelling cannula over the puncture cannula into the vessel.

### DETAILED DESCRIPTION OF THE INVENTION

The introduction device shown in the Figures comprises a pistol-shaped housing 1, with a grip part 2 for handling the device. The housing 1 at the top blends into a gun-barrel-shaped part 3, whose distal end 4 comprises a contact surface 5.

On the upper side of the gun-barrel-like part 3 there is provided a telescope-like guide 6 which accommodates two rods 7 which are arranged at a distance and parallel to one another and which are traversable in the axis direction of the rods 7 by way of an electromagnetic drive which is not shown in detail. At the distal end of the rods 7 there is provided a holder 8 for the releasable accommodation of a puncture cannula 9.

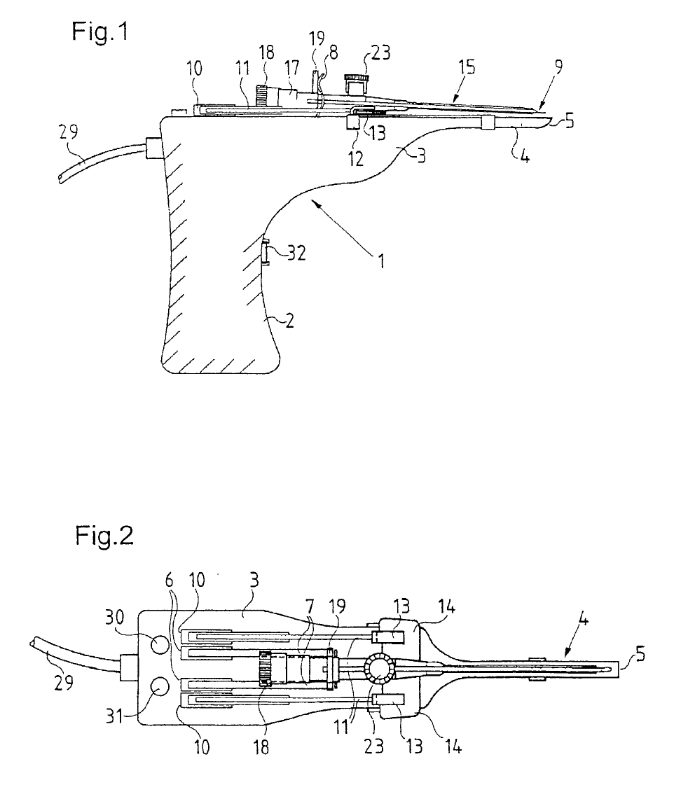

Parallel to the guide 6 on the upper side of the gun-barrel-like part 3 there is provided a further guide whose likewise telescopic rods 11 are arranged parallel to one another and parallel to the rods 7. The rods 11, as indicated in FIG. 1, comprise lugs 12 which by way of longitudinal reliefs are connected to an electromotoric drive likewise arranged within the grip part. At the distal ends of the rods 11 there are provided -like holders 13 with which the fins 14 of an indwelling cannular 15 are held.

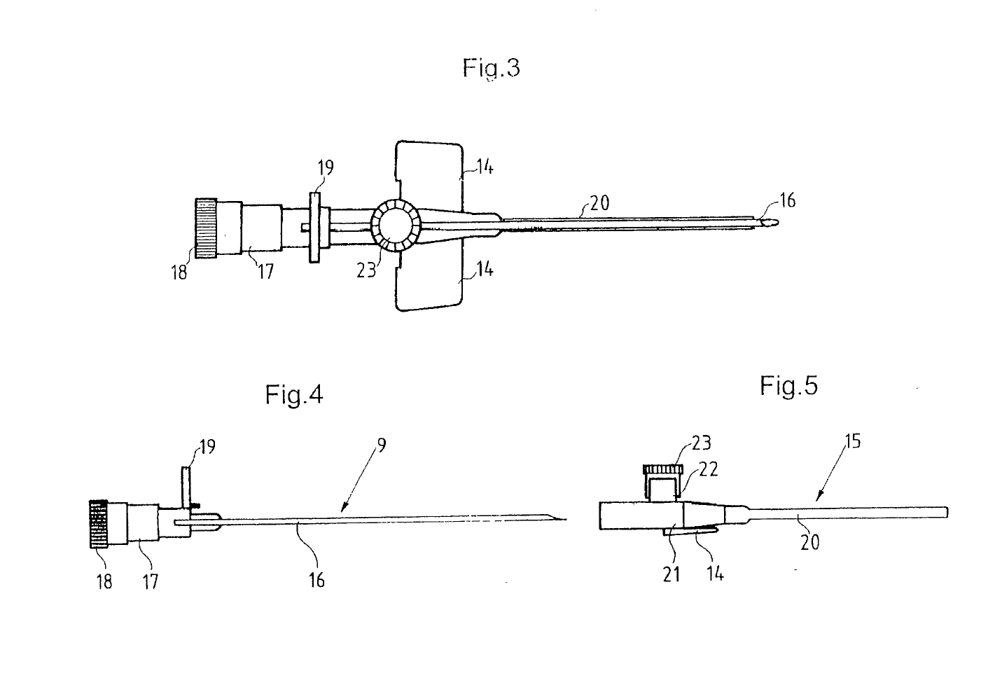

With the cannulas 9, 15 shown in the figures it is the case of common indwelling cannulas, as are on the market under the trademark VENFLON of the company VIGGO AG, Sweden and are shown by way of FIGS. 3 to 5 in detail. Such a cannula arrangement consists of an essentially rigid puncture cannula 9 as is shown in FIG. 4, and of an elastically bendable plastic indwelling cannula 15 as is shown in FIG. 5. The puncture cannula consists of a common metal tube 16 ground off obliquely on the distal side, whose proximal end is held in a plastic part 17 which on the proximal side is designed as a Luer connection and is closed off by way of a closure plug 18. On the plastic part 17 there upwardly projects a guide part 19, it serves the manual removal of the puncture cannula from the indwelling cannula after the puncture of the vessel has been effected.

The bending-elastic indwelling cannula 15 consists of a flexible plastic tube 20 which on the proximal side likewise opens into a plastic part 21, whose proximal end is also designed as a Luer connection. Furthermore the plastic part 21 further comprises a transverse access 22 which may be closed off by way of a closure plug 23. The cannulas 9 and 15 are dimensioned such that in the assembled form they form the arrangement shown in FIG. 3, in which the distal, ground-off free end of the metal tube 16 projects beyond the distal free end of the plastic tube 20.

The cannulas 9 and 15 in the arrangement shown in FIG. 3 are fastened in a clamped manner in the device, as this is represented by way of FIGS. 1 and 2. With this the guide 19 of the cannula 9 is fastened in the holder 8 and the fins 14 are fastened in the holders 13. The rods 7 and 11 when they are completely retracted in the initial situation, as this is shown in the FIGS. 1 and 2, are designed such that the distal end of the metal tube 16 ends in front of the contact surface 5, thus in front of the distal end of the device. The cannulas 9 and 15 are at the same time only fastened in the holders 8 and 13, otherwise however they are arranged at a distance to the housing in order to ensure sterility.

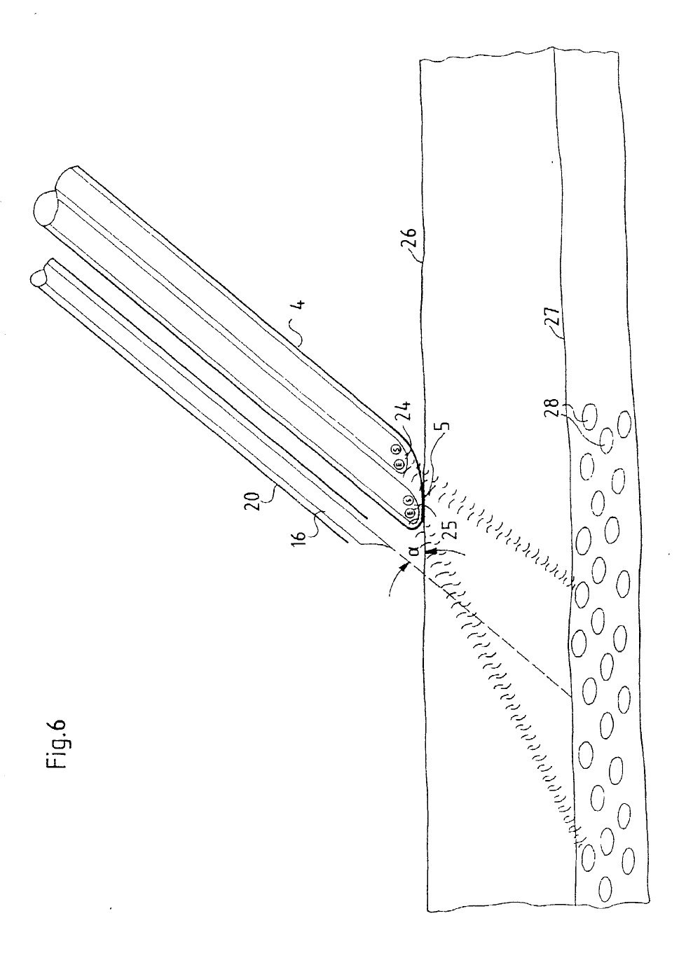

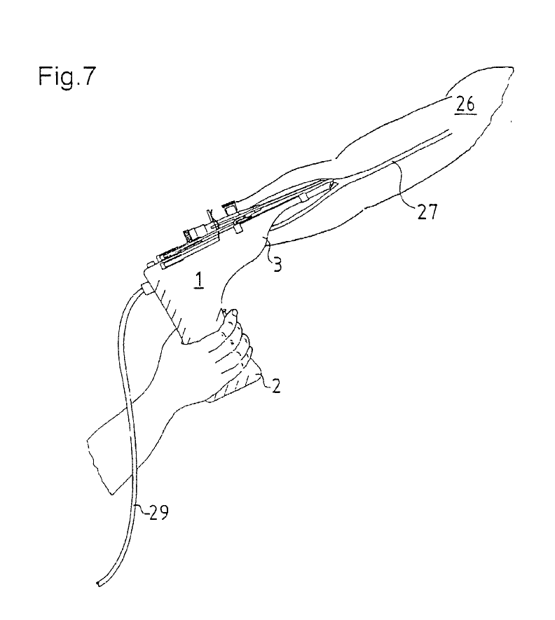

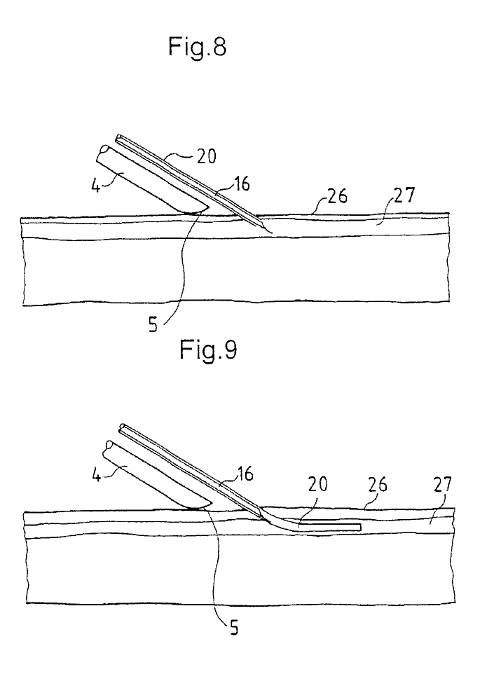

Directly behind the contact surface 5 there are arranged two ultrasound locating means 24 and 25 within the housing 1, as this is schematically shown in FIG. 6. The locating means consist either, as in the embodiment shown, in each case of an ultrasound emitter S and an ultrasound receiver E, or of a combination of emitter and receiver (pulse operation). With this the received reflection signal of the ultrasound waves are not only evaluated with regard to temporal intervals of the returning signals as with the usual ultrasound location, but furthermore also with regard to the frequency shifting which results according to the Doppler effect when ultrasound waves are reflected on moved bodies, in particular the blood cells of a blood vessel. This is schematically shown in FIG. 6, with this the surface of the skin is indicated at 26, the vessel to be located at 27 and the blood cells flowing therein at 28. The ultrasound locating means 24 and 25 are aligned such that they not only detect the expected puncture location of the puncture cannula 9 with the vessel 27, but also the region directly in front of and behind this location, so that the course of the vessel may be represented. The evaluation of the signals of the ultrasound locating means 24 and 25 is effected via a control which is not shown in the figures and which is arranged within the housing 1. The voltage supply of the apparatus is effected via a mains cable 29 or an incorporated energy storer (e.g. batteries). Within the pistol-shaped housing there are furthermore located the electrical drive required for traversing the rods 7 and 11, with where appropriate mechanical gears connected intermediately.

The distal end of the gun-barrel-like part 3 is closed of by the contact surface 5 which as is shown in FIG. 6, is formed slightly crowned in order on the one hand to permit an easy alignment of the housing 1 with regard to the surface of the skin 26. On the other hand however the contact surface 5 is formed flattened such that on applying this on the surface of the skin 26 there is provided a secure hold at an angular position which roughly corresponds to the expected optimal angular position. This angle α (angle between traversing direction of the cannulas 9, 15 and the contact surface) is 35° in the embodiment example, and usually lies between 30° and 45°, according to application.

Before the beginning of the introduction procedure the apparatus is equipped with a cannula arrangement according to FIG. 3 so that there results the initial situation previously described and shown by way of the FIGS. 1 and 2. The apparatus detects by way of a bar code reader arranged on its upper side (not shown in the figures), where the fins 14 of the indwelling cannula 15 are arranged after application into the associated holder, detects a bar code located on the fins 14. On account of information coded in this bar code, the type and the size of the applied cannula is transmitted to- the control.

For the introduction of the cannula, the operating person grips the grip part 2 of the housing with the hand and places the distal end 4 with the contact surface onto the skin surface 16 where thereunder there is to be expected a course of a vessel. On account of the Doppler evaluation the location means 24 and 25 may locate blood vessels. As soon as a blood vessel has been detected by one of the two locating means 24 or 25 a first light diode 30 lights up on the upper side of the housing. As long as this diode 30 does not light up one must traverse the contact surface 5 on the skin surface 2 until a suitable vessel 27 has been located. If the light diode 30 lights up, the apparatus 1 must merely be brought into the angular position and direction envisaged for introducing the needle. This is the case when both locating means 24 and 25 detect the same vessel and the apparatus is located at designated angles to the vessel 27. A second light diode 31 then lights up, simultaneously a release button 32 is switched free so that the programm-controlled introduction of the cannulas may be activated.

Thus if the operating person by way of the signal of the light diodes 30 and 31 recognises that the designated position with regard to the vessel 27 to be punctured has been reached, the release button 32 is actuated. Thereafter firstly the rods 7 and 11 are simultaneously extended out, by which means the surface of the skin is pierced. The rods 7 and 11 under the control of the locating means 24 and 25 are extended until the distal end of the puncture cannula 9 as well as the distal end of the indwelling cannula 15 have reached the inside of the vessel 27. Then the rods 11 are further extended out, the rods 7 however are simultaneously or subsequently retracted (see FIG. 9). The puncture cannnula 9 by way of the rods is retracted so far that it just closes off the indwelling cannula 15 on the proximal side.

The further removal of the puncture cannula 9 is effected manually, since then the indwelling cannula on the proximal side must anyway be provided with a closure plug or connected. It is to be understood that the holders 8 and 13 are likewise to be released in order to separate the apparatus from the cannulas. In a further embodiment this may likewise be part of the program control. In the embodiment shown the holders however are designed as clamping holders so that they may be released with the free hand with a slight force effort.

It is to be understood that with the previously described device not only indwelling cannulas, but also puncture cannulas such as are for example directly connected to syringes, may be brought into a vessel. Basically an empirical evaluation of the exit signals of the locating means 24 and 25 is sufficient in order to permit a secure control of the device and to achieve the designated puncture of the vessel. Here also apart from the described simple signal transmitters 30 and 31 one may provide acoustic signal transmitters as locating aids as well as complex optical aids, for example in the form of a co-ordinate cross with four light diodes at the ends for a simplified positioning. For particularly complicated operations in deeper-lying vessels an additional monitor control may also be provided, as is known for locating means of the previously described type.

1. A device for introducing an elastically bendable indwelling cannula together with an essentially rigid puncture cannula located therein, into a blood vessel of a human or animal body with the following features:
at least one locating means for locating the vessel and for detecting the course of the vessel in front of and behind the puncture location or the expected puncture location of the puncture cannula,
at least one guide for the cannulas to be introduced,
holders with which the cannulas are fastenable on the device, and which are mounted displaceably in the introduction direction as well as in the counter direction,
drive means for moving the holders along the guide in a manner such that the indwelling cannula is traversable separate from the puncture cannula,
a control which activates the drive means for introducing the distal cannula ends into the vessel in dependence on the exit signal of the locating means, as well as for the subsequent retraction of the puncture cannula from the indwelling cannula.

2. A device according to claim 1, wherein there are provided two preferably Doppler-sonographically functioning locating means of which one detects in the region in front of and the other in the region behind the puncture location or the puncture location to be expected.

3. A device according to one of the preceding claims, wherein the control releases the drive moving the cannulas in the introduction direction only when the evaluation of the signals emitted by the locating means results in that the device is located in a position to the vessel to be punctured, which is suitable for the puncture procedure.

4. A device according to one of the preceding claims, wherein there is provided an optical and/or acoustic display which emits a signal when the device is situated in the designated position for the puncture of the located vessel.

5. A device according to one of the preceding claims, wherein the distal end of the device comprises a contact surface which is preferably formed crowned and which is set obliquely with respect to the movement direction of the cannula holder, and specifically preferably at an angle between 30° and 45°.

6. A device according to one of the preceding claims, wherein there is provided a linear guide for the puncture cannula and a further linear guide parallel to this for the indwelling cannula.

7. A device according to one of the preceding claims, wherein the device is formed essentially in the shape of a pistol and comprises a release button in the front part of the grip piece, wherein the control and evaluation electronics are preferably arranged within the grip piece.

8. A device according to one of the preceding claims, wherein to each guide there is allocated an electronic drive in the form of an electric motor which is preferably arranged within the grip part.

9. A device according to one of the preceding claims, wherein there are provided means for detecting the cannula type and/or size.

10. A device according to one of the preceding claims, wherein the electromotors are activated by the control such that firstly the indwelling cannula together with the puncture cannula is traversed distally, wherein after puncture of the vessel and after penetration of the vessel wall by the indwelling cannula this is traversed further distally and the puncture cannula is simultaneously or subsequently retracted.
